(19) United States
(12) Patent Application Publication
(10) Pub. No.: US 2004/0205906 A1
(43) Pub. Date: Oct. 21, 2004

(54) Dyeing composition comprising an acylaminophenol coupler and use of this coupler for dyeing keratinous fibres

(76) Inventors: Laurent Vidal, Paris (FR); Alain Lagrange, Coupvray (FR)

Correspondence Address: FINNEGAN, HENDERSON, FARABOW,, GARRETT & DUNNER, L.L.P., 1300 I Street, N.W., Washington, DC, 20005-3315, US

(21) Appl. No.: 10/758,215

(22) Filed: Jan. 16, 2004

Related U.S. Application Data
(60) Provisional application No. 60/464,856, filed on Apr. 24, 2003.

(30) Foreign Application Priority Data
Jan. 17, 2003 (FR) 03 00540

(51) Int. Cl. A61K 7/13

(52) U.S. Cl. 8/405

(57) ABSTRACT

Disclosed herein is a dyeing composition comprising an acylaminophenol coupler and a method for dyeing keratinous fibres, such as hair, using this coupler.

This application claims benefit of U.S. Provisional Application No. 60/464,856, filed Apr. 24, 2003, and French Application 03 00540 filed Jan. 17, 2003.

Disclosed herein, for example, is a novel dyeing composition comprising a acylaminophenol coupler, the use of this composition for dyeing keratinous fibres, and a method for dyeing keratinous fibres using this composition.

It is known to dye keratinous fibres and in particular human hair with dyeing compositions containing oxidation dye precursors, generally called oxidation bases, in particular ortho- or para-phenylenediamines, ortho- or para-aminophenols, or heterocyclic compounds such as diaminopyrazole derivatives, pyrazolo[1,5-a]pyrimidine derivatives, pyrimidine derivatives, pyridine derivatives, 5,6-dihydroxyindole derivatives, and 5,6-dihydroxyindoline derivatives. Oxidation dye precursors or oxidation bases are colourless or weakly coloured compounds which, when combined with oxidizing products, can give rise, through a process of oxidative condensation, to coloured or colouring compounds.

It is also known that it is possible to vary the shades obtained with these oxidation bases by combining them with couplers or colour modifiers, the latter being chosen in particular from aromatic meta-diamines, meta-aminophenols, meta-hydroxyphenols and certain heterocyclic compounds such as, for example, pyrazolo[1,5-b]-1,2,4-triazole derivatives, pyrazolo[3,2-c]-1,2,4-triazole derivatives, pyrazolo[1,5-a]pyrimidine derivatives, pyridine derivatives, pyrazol-5-one derivatives, indoline derivatives or indole derivatives.

The variety of molecules used in oxidation bases and couplers allows a rich pallet of colours to be obtained.

In one embodiment, it is desirable that the so-called “permanent” colour obtained using these oxidation dyes satisfy a number of requirements. Desirable properties include possibly obtaining shades in the desired intensity, exhibiting good resistance towards external agents (light, adverse weather conditions, washing, permanent waving, perspiration, rubbing), and being non-toxic.

The dyes may also make it possible to cover grey hair, and may be the least selective possible. That is to say the dyes may make it possible to obtain the smallest possible differences in colour right along the same keratinous fibre, which may indeed be differently sensitive to the dyes between its tip and its root. The dyes may also have good chemical stability in the formulations.

There have already been proposed, in particular in Patent Applications WO 98/52519, JP2521636, FR-A-1 596 879 or FR-A-2 233 984, oxidation dyeing compositions containing certain N-(2-hydroxy-4-aminophenyl)acetamide derivatives as couplers, in combination with oxidation bases conventionally used in oxidation dyeing, such as, for example, para-phenylenediamines, para-aminophenols or certain pyridines.

Such compositions are, however, not always satisfactory, in particular from the point of view of the intensity of the colours obtained. Furthermore, the colours obtained on hair by combining such couplers with para-phenylenediamine derivatives may not always be stable under the action of light and exhibit an unaesthetic change in colour.

One aspect of the present disclosure is to develop novel dyeing compositions not having the disadvantages of the prior art compositions. For example, one aspect of the disclosure is to develop novel couplers having properties such that the compositions containing them may confer on hair at least one of the following properties: colour intensity, variety of shades, uniformity of colour and fastness towards various external attacks to which the hair may be subjected.

One embodiment of the present disclosure is a dyeing composition, comprising at least one cosmetic medium appropriate for dyeing keratinous fibres, at least one oxidation base and at least one coupler chosen from compounds of the following formula (I) and any corresponding acid and base addition salts thereof

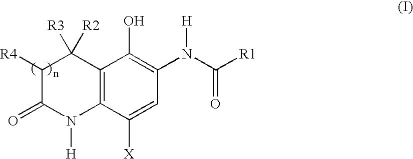

wherein

R₁ is a substituted or unsubstitued alkyl radical comprising from 1 to 10 carbon atoms, wherein the substituted alkyl radical comprises at least one radical chosen from halogen atoms, hydroxyl radicals, alkoxy radicals, carboxyl radicals, (C₁-C₄)alkylcarboxamido radicals, (NH₂—SO₂—) sulphonamido radicals, (C₁-C₄)alkylsulphonamido radicals, and NR₁₁R₁₂, wherein R₁₁ and R₁₂, which may be the same or different, are chosen from hydrogen atoms, and (C₁-C₂)alkyl radicals optionally substituted with at least one substituent chosen from hydroxyls and (C₁-C₂)alkoxys;

R₂, R₃ and R₄, which may be the same or different, are chosen from

(i) hydrogen atoms, and

(ii) (C₁-C₄)alkyl radicals optionally substituted with at least one radical chosen from hydroxyl, alkoxy, carboxyl, (C₁-C₄)alkylcarboxamido (AlkylNHCO—), (C₁-C₄)alkylsulphonyl (AlkSO₂—), (C₁-C₄)alkylsulphoxide (AlkylSO—), sulphonamido (NH₂SO₂—), (C₁-C₄)alkylsulphonamido (AlkylNHSO₂—),sulphonic (—SO₃H), and NR₁₁R₁₂ radicals,

X is a hydrogen atom, a halogen atom, a substituted or unsubstituted (C₁-C₄)alkoxy radical, or a substituted or unsubstituted aryloxy radical, and

n is 0 or 1.

One aspect of the disclosure is the use of the couplers of formula (I) for dyeing keratinous fibres, such as hair, and the method for dyeing keratinous fibres.

Another aspect of the disclosure is a method for dyeing, a kit for dyeing and a device for dyeing keratinous fibres using the composition disclosed herein.

In the context of the disclosure, the expression alkyl is understood to mean linear or branched radicals.

In one emodiment, R₁ is chosen from a methyl, ethyl, isopropyl, methoxymethyl, hydroxymethyl, 1-carboxymethyl, 1-aminomethyl, 2-carboxyethyl, 2-hydroxyethyl, 3-hydroxypropyl, 1,2-dihydroxyethyl, 1-hydroxy-2-aminoethyl and 2-hydroxy-1-aminoethyl radical.

For example, R₁ may also be chosen from methyl, ethyl, hydroxymethyl, 2-hydroxyethyl and 1,2-dihydroxyethyl radicals. Further for example, R₁ is chosen from a methyl, ethyl, propyl, hydroxymethyl and 2-hydroxyethyl radical.

According to one embodiment, R₁ is an unsubstituted alkyl radical, for example methyl, ethyl or propyl.

In one embodiment, R₂, R₃ and R₄, which may be identical or different, are chosen from hydrogen atoms and C₁-C₄ alkyl radicals optionally substituted with at least one radical chosen from hydroxyl, methoxy, ethoxy, carboxyl, (C₁-C₄)alkylcarboxamido, and sulphonamido (NH₂—SO₂) radicals. According to another embodiment, R₂, R₃ and R₄ are chosen from hydrogen atoms and unsubstituted alkyl radicals.

In one embodiment, when X is a substituted alkoxy or aryloxy radical, the substituents may be, for example, conventional radicals chosen from hydroxyl, amino, halogen, alkoxy and amido radicals, and the like. For example, X may be chosen from a hydrogen atom, a chlorine atom and an alkoxy, for example methoxy, radical.

According to one embodiment disclosed herein, the coupler corresponds to the formula (I′)

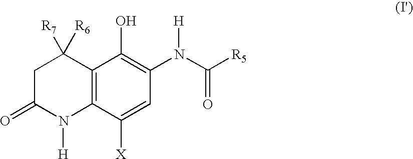

wherein R₅ is a C₁-C₆ alkyl radical, X is a hydrogen atom, a halogen atom or an alkoxy radical, and R₆ and R₇, which may be identical or different, are chosen from methyl radicals and ethyl radicals.

Non-limiting examples of the couplers of formula (I) or (I′) include:

N-(8-chloro-5-hydroxy-4,4-dimethyl-2-oxo-1,2,3,4-tetrahydroquinolin-6-yl)acetamide,

N-(5-hydroxy-4,4-dimethyl-2-oxo-1,2,3,4-tetrahydroquinolin-6-yl)acetamide,

N-(8-chloro-5-hydroxy-4,4-dimethyl-2-oxo-1,2,3,4-tetrahydroquinolin-6-yl)propylamide,

N-(5-hydroxy-4,4-dimethyl-2-oxo-1,2,3,4-tetrahydroquinolin-6-yl)propylamide,

N-(8-methoxy-5-hydroxy-4,4-dimethyl-2-oxo-1,2,3,4-tetrahydroquinolin-6-yl)acetamide,

N-(8-methoxy-5-hydroxy-4,4-dimethyl-2-oxo-1,2,3,4-tetrahydroquinolin-6-yl)propylamide,

N-(8-chloro-5-hydroxy-2-oxo-1,2,3,4-tetrahydroquinolin-6-yl)acetamide,

N-(5-hydroxy-2-oxo-1,2,3,4-tetrahydroquinolin-6-yl)acetamide,

N-(8-chloro-5-hydroxy-2-oxo-1,2,3,4-tetrahydroquinolin-6-yl)propylamide,

N-(5-hydroxy-2-oxo-1,2,3,4-tetrahydroquinolin-6-yl)propylamide,

N-(8-methoxy-5-hydroxy-2-oxo-1,2,3,4-tetrahydroquinolin-6-yl)acetamide,

N-(8-methoxy-5-hydroxy-2-oxo-1,2,3,4-tetrahydroquinolin-6-yl)propylamide,

N-(7-chloro-4-hydroxy-3,3-dimethyl-2-oxo-2,3-dihydroindol-5-yl)acetamide,

N-(7-methoxy-4-hydroxy-3,3-dimethyl-2-oxo-2,3-dihydroindol-5-yl)acetamide,

N-(4-hydroxy-3,3-dimethyl-2-oxo-2,3-dihydroindol-5-yl)acetamide,

N-(7-chloro-4-hydroxy-3,3-dimethyl-2-oxo-2,3-dihydroindol-5-yl)propylamide,

N-(7-methoxy-4-hydroxy-3,3-dimethyl-2-oxo-2,3-dihydroindol-5-yl)propylamide,

N-(4-hydroxy-3,3-dimethyl-2-oxo-2,3-dihydroindol-5-yl)propylamide,

N-(7-chloro-4-hydroxy-2-oxo-2,3-dihydroindol-5-yl)acetamide,

N-(7-methoxy-4-hydroxy-2-oxo-2,3-dihydroindol-5-yl)acetamide,

N-(4-hydroxy-2-oxo-2,3-dihydroindol-5-yl)acetamide,

N-(7-chloro-4-hydroxy-2-oxo-2,3-dihydroindol-5-yl)propylamide,

N-(7-methoxy-4-hydroxy-2-oxo-2,3-dihydroindol-5-yl)propylamide, and

N-(4-hydroxy-2-oxo-2,3-dihydroindol-5-yl)propylamide

For example, couplers may also be chosen from N-(8-chloro-5-hydroxy-4,4-dimethyl-2-oxo-1,2,3,4-tetrahydroquinolin-6-yl)acetamide, N-(5-hydroxy-4,4-dimethyl-2-oxo-1,2,3,4-tetrahydroquinolin-6-yl)acetamide, N-(8-chloro-5-hydroxy-4,4-dimethyl-2-oxo-1,2,3,4-tetrahydroquinolin-6-yl)propylamide, N-(5-hydroxy-4,4-dimethyl-2-oxo-1,2,3,4-tetrahydroquinolin-6-yl)propylamide, N-(8-methoxy-5-hydroxy-4,4-dimethyl-2-oxo-1,2,3,4-tetrahydroquinolin-6-yl)acetamide, N-(8-methoxy-5-hydroxy-4,4-dimethyl-2-oxo-1,2,3,4-tetrahydroquinolin-6-yl)propylamide, N-(8-chloro-5-hydroxy-2-oxo-1,2,3,4-tetrahydroquinolin-6-yl)acetamide, N-(5-hydroxy-2-oxo-1,2,3,4-tetrahydroquinolin-6-yl)acetamide, N-(8-chloro-5-hydroxy-2-oxo-1,2,3,4-tetrahydroquinolin-6-yl)propylamide, N-(5-hydroxy-2-oxo-1,2,3,4-tetrahydroquinolin-6-yl)propylamide, N-(8-methoxy-5-hydroxy-2-oxo-1,2,3,4-tetrahydroquinolin-6-yl)acetamide, and N-(8-methoxy-5-hydroxy-2-oxo-1,2,3,4-tetrahydroquinolin-6-yl)propylamide.

The molecules corresponding to general formula (I) with n=1 may, for example, be synthesized according to the synthesis protocols described in U.S. Pat. No. 4,564,586 and the references cited therein.

For example, 8-chloro-5-hydroxy-4,4-dimethyl-6-amino-3,4-dihydro-2(1H)-quinoline 1 described in that patent may be used as a reagent for the synthesis of the 6-amido derivatives of general formula (I) by conventional reaction with the acid chloride R₆—COCl in an aprotic solvent.

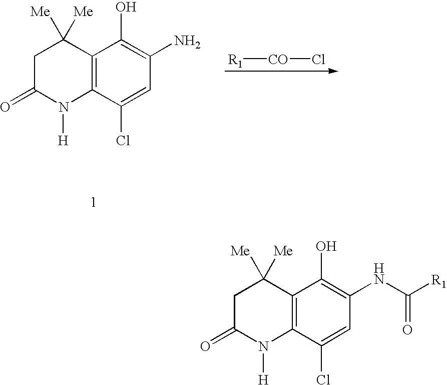

The molecules corresponding to general formula (I) with n=0 may, for example, be synthesized according to the synthesis protocols described in U.S. Pat. No. 4,904,575. For example, the 5-amino-7-chloro-3,3-dimethyl-4-hydroxyoxindole hydrochloride 2 described in this patent may be used as a reagent for the synthesis of the 5-amido derivatives of general formula (I) by conventional reaction with the acid chloride R₆—COCl in an aprotic solvent.

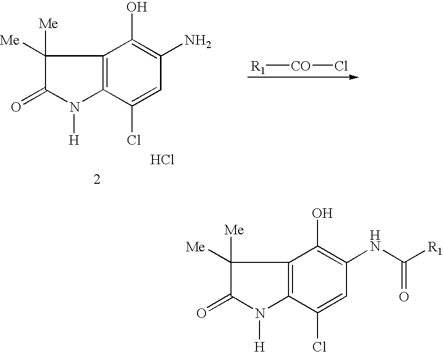

The dyeing composition disclosed herein may also comprise at least one oxidation base.

In one embodiment, the at least one oxidation base may be any conventional oxidation base in the keratinous fibre dyeing field. By way of non-limiting example, these oxidation bases may be chosen from para-phenylenediamines, bisphenylalkylenediamines, para-aminophenols, bis-para-aminophenols ortho-aminophenols, heterocyclic bases and the addition salts thereof.

Among the para-phenylenediamines, there may be mentioned, by way of non-limiting example, para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl-β-hydroxyethyl)-para-phenylenediamine, N—(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene, 3-hydroxy-1-(4′-aminophenyl)pyrrolidine and the acid addition salts thereof.

Among the para-phenylenediamines mentioned above, mention may be made, for example, of para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, 2-β-acetylaminoethyloxy-para-phenylenediamine, and the acid addition salts thereof.

Among the bisphenylalkylenediamines, there may be mentioned, by way of example, N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)-tetramethylenediamine, N,N′-bis(β-hydroxyethyl)-N,N′-bis(4-aminophenyl)tetramethylene-diamine, N,N′-bis(4-methylaminophenyl)tetramethylenediamine, N,N′-bis(ethyl)-N,N′-bis(4′-amino-3′-methylphenyl)ethylenediamine, 1,8-bis(2,5-diaminophenoxy)-3,6-dioxaoctane, and the acid addition salts thereof.

Among the para-aminophenols, there may be mentioned, by way of example, para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-fluorophenol, and the acid addition salts thereof.

Among the ortho-aminophenols, there may be mentioned, by way of example, 2-aminophenol, 2-amino-5-methylphenol, 2-amino-6-methylphenol, 5-acetamido-2-aminophenol, and the acid addition salts thereof.

Among the heterocyclic bases, there may be mentioned, by way of example, pyridine derivatives, pyrimidine derivatives and pyrazole derivatives.

Among the pyridine derivatives, there may be mentioned, for example, the compounds described in Patents GB 1,026,978 and GB 1,153,196, such as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine, 3,4-diaminopyridine, and the acid addition salts thereof.

Other non-limiting examples of pyridine oxidation bases disclosed herein are 3-aminopyrazolo[1,5-a]pyridines or their addition salts which are described in Patent Application FR 2801308. By way of non-limiting example, there may be mentioned pyrazolo[1,5-a]pyridin-3-ylamine; 2-acetylaminopyrazolo[1,5-a]pyridin-3-ylamine; 2-morpholin-4-ylpyrazolo[1,5-a]pyridin-3-ylamine; 3-aminopyrazolo[1,5-a]pyridine-2-carboxylic acid; 2-methoxypyrazolo[1,5-a]pyridin-3-ylamino; (3-aminopyrazolo[1,5-a]pyridin-7-yl)methanol; 2-(3-aminopyrazolo[1,5-a]pyridin-5-yl)methanol; 2-(3-aminopyrazolo[1,5-a]pyridin-7-yl)ethanol; (3-aminopyrazolo[1,5-a]pyridin-2-yl)methanol; 3,6-diaminopyrazolo[1,5-a]pyridine; 3,4-diaminopyrazolo[1,5-a]pyridine; pyrazolo[1,5-a]pyridine-3,7-diamine; 7-morpholin-4-ylpyrazolo[1,5-a]pyridin-3-ylamine; pyrazolo[1,5-a]pyridine-3,5-diamine; 5-morpholin-4-ylpyrazolo[1,5-a]pyridin-3-ylamine; 2-[(3-aminopyrazol[1,5-a]pyridin-5-yl)(2-hydroxyethyl)amino]ethanol; 2-[(3-aminopyrazolo[1,5-a]pyridin-7-yl)(2-hydroxyethyl)amino]ethanol, 3-aminopyrazolo[1,5-a]pyridin-5-ol; 3-aminopyrazolo[1,5-a]pyridin-5-ol, 3-aminopyrazolo[1,5-a]pyridin-6-ol; 3-aminopyrazolo[1,5-a]pyridin-7-ol; and the acid and base addition salts thereof.

Among the pyrimidine derivatives, there may be mentioned, for example, the compounds described in Patents DE 2,359,399; JP 88-169,571; JP 05-63124; EP 0770375 or Patent Application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine, and the pyrazolopyrimidine derivatives. Among these pyrazolopyrimidine derivatives non-limiting mention may be made of those described in Patent Application FR-A-2,750,048, such as pyrazolo[1,5-a]pyrimidine-3,7-diamine; 2,5-dimethylpyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl)(2-hydroxyethyl)amino]ethanol, 2-[(7-aminopyrazolo[1,5-a]pyrimidin-3-yl)-(2-hydroxyethyl)amino]ethanol, 5,6-dimethylpyrazolo[1,5-a]pyrimidine-3,7-diamine, 2,6-dimethylpyrazolo[1,5-a]pyrimidine-3,7-diamine, 2,5,N7,N7-tetramethylpyrazolo[1,5-a]pyrimidine-3,7-diamine, 3-amino-5-methyl-7-imidazolylpropylaminopyrazolo[1,5-a]pyrimidine and the acid addition salts thereof and their tautomeric forms, when a tautomeric equilibrium exists.

Among the pyrazole derivatives, there may be mentioned, for example, the compounds described in Patents DE 3,843,892, DE 4,133,957 and Patent Applications WO 94/08969, WO 94/08970, FR-A-2,733,749 and DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethyl-pyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole, and the acid addition salts thereof.

In one embodiment, the at least one oxidation base present in the composition of the invention is generally present in an amount ranging from 0.001% to 10% by weight relative to the total weight of the dyeing composition, for example ranging from 0.005% to 6%.

The dyeing composition disclosed herein may further comprise at least one additional coupler conventionally used for dyeing keratinous fibres, other than those of formula (I). Among these couplers, there may be mentioned, for example, meta-phenylenediamines, meta-aminophenols, meta-diphenols, naphthalene couplers, heterocyclic couplers and the addition salts thereof.

By way of non-limiting example, there may be mentioned 2-methyl-5-aminophenol, 5-N-(β-hydroxyethyl)amino-2-methylphenol, 6-chloro-2-methyl-5-aminophenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene, 2,6-bis(β-hydroxyethylamino)toluene and the acid addition salts thereof.

In one embodiment of the composition disclosed herein, the at least one coupler is generally present in an amount ranging from 0.001% to 10% by weight relative to the total weight of the dyeing composition, for example, ranging from 0.005% to 6%.

In general, the addition salts of the oxidation bases and couplers disclosed herein may be chosen from, for example, the addition salts with an acid such as the hydrochlorides, hydrobromides, sulphates, citrates, succinates, tartrates, lactates, tosylates, benzenesulphonates, phosphates and acetates and the addition salts with a base such as sodium hydroxide, potassium hydroxide, ammonium hydroxide, amines and alkanolamines.

In one embodiment, the dyeing composition disclosed herein may comprise at least one direct dye which may be chosen from, for example nitro dyes of the benzene series, azo direct dyes and methinic direct dyes. These direct dyes may be of a nonionic, anionic or cationic nature.

In one embodiment, the cosmetic medium appropriate for dyeing, also called dye carrier, comprises water or of a mixture of water and at least one organic solvent for solubilizing the compounds which might not be sufficiently soluble in water. As the organic solvent, mention may be made of, for example, lower C₁-C₄ alkanols such as ethanol and isopropanol; polyols and polyol ethers such as 2-butoxyethanol, propylene glycol, propylene glycol monomethyl ether, diethylene glycol monoethyl ether and monomethyl ether; and aromatic alcohols such as benzyl alcohol or phenoxethanol; and mixtures thereof.

The solvents may be present, for example, in amounts ranging from 1% to 40% by weight, such as from 5% to 30% by weight, relative to the total weight of the dyeing composition.

The dyeing composition disclosed herein may further comprise, for example, various adjuvants conventionally used in hair dyeing compositions, such as anionic, cationic, nonionic, amphoteric or zwitterionic surfactants or mixtures thereof; anionic, cationic, nonionic, amphoteric or zwitterionic polymers or mixtures thereof; inorganic or organic thickeners, for example, the associative thickeners such as anionic, cationic, nonionic and amphoteric polymers; antioxidants; penetrating agents; sequestrants; perfumes; buffers; dispersing agents; conditioning agents such as, for example, modified or unmodified, volatile or nonvolatile silicones; film-forming agents; ceramides; preservatives; and opacifying agents.

The above adjuvants may, for example, each be present in an amount ranging from 0.01% to 20% by weight relative to the total weight of the composition.

Of course, persons skilled in the art will be careful to choose this or these optional additional compounds such that the advantageous properties intrinsically attached to the oxidation dyeing composition disclosed herein are not substantially impaired by the addition(s) envisaged.

In one embodiment, the pH of the dyeing composition in accordance with the invention generally ranges from approximately 3 to 12, for example, from approximately 5 to 11. It may, for example, be adjusted to the desired value by means of acidifying or alkalizing agents customarily used in dyeing keratinous fibres or with the aid of conventional buffering systems.

Among the acidifying agents, there may be mentioned, by way of example, inorganic and organic acids such as hydrochloric acid, orthophosphoric acid, sulphuric acid, carboxylic acids such as acetic acid, tartaric acid, citric acid, lactic acid, and sulphonic acids.

Among the alkalizing agents, there may be mentioned, by way of example, ammonium hydroxide, alkali metal carbonates, alkanolamines such as mono-, di- and triethanolamines and their derivatives, sodium or potassium hydroxides and the compounds of the following formula (II):

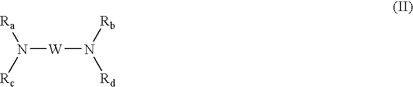

in which W is a propylene residue optionally substituted with a substituent chosen from hydroxyl groups and C₁-C₄ alkyl radicals; Rₐ, R_{b}, R_{c} and R_{d}, which may be identical or different, are chosen from hydrogen atoms, C₁-C₄ alkyl radicals and C₁-C₄ hydroxyalkyl radicals.

The dyeing composition disclosed herein may, for example, be provided in various forms, such as in the form of liquids, creams or gels, or in any other appropriate form for dyeing keratinous fibres, such as human hair.

The method disclosed herein is a method in which the composition according to the present disclosure, as defined above, is applied to the keratinous fibres in the presence of an oxidizing agent for a sufficient time to develop a desired colour. In one embodiment, the colour may be developed at an acidic, neutral or alkaline pH and the oxidizing agent may be added to the composition disclosed herein just at the time of use or it can be used from an oxidizing composition comprising it, applied simultaneously or sequentially with the composition dislosed herein.

According to one non-limiting embodiment, the composition disclosed herein is mixed, for example, at the time of use, with a composition comprising, in a medium appropriate for dyeing, at least one oxidizing agent, this oxidizing agent being present in a sufficient quantity to develop a colour. The mixture obtained is then applied to the keratinous fibres. After an exposure time ranging from 3 to 50 minutes, for example, 5 to 30 minutes, the keratinous fibres are rinsed, washed with shampoo, rinsed again and then dried.

The oxidizing agents conventionally used for the oxidation dyeing of keratinous fibres are, for example, hydrogen peroxide, urea peroxide, alkali metal bromates, persalts such as perborates and persulphates, peracids and the oxidase enzymes, among which there may be mentioned peroxidases, oxidoreductases with 2 electrons such as uricases and oxygenases with 4 electrons such as laccases. In one non-limiting embodiment, hydrogen peroxide may be used.

The oxidizing composition may further comprise various adjuvants conventionally used in hair dyeing compositions and as defined above.

The pH of the oxidizing composition comprising the oxidizing agent is such that, after mixing with the dyeing composition, the pH of the resulting composition applied to keratinous fibres varies, for example, from 3 to 12, such as from 5 to 11. It may be adjusted to the desired value by means of acidifying or alkalizing agents customarily used for dyeing keratinous fibres and as defined above.

One embodiment of the disclosure is a ready-to-use composition comprising at least one dyeing composition, as disclosed herein, and at least one oxidizing agent. The ready-to-use composition which is finally applied to the keratinous fibres may be provided, for example, in various forms, such as in the form of liquids, creams or gels, or in any other form appropriate for dyeing keratinous fibres such as human hair.

Another aspect of the disclosure is also a dyeing kit which comprises a first composition comprising at least one oxidation base and at least one coupler of formula (I) and a second composition which comprises an oxidizing agent.

An additional aspect of the disclosure is a multicompartment device or dyeing “kit” in which a first compartment comprises at least one dyeing composition as defined above and a second compartment comprises at least one oxidizing composition. This device may be equipped with means which make it possible to deliver the desired mixture to the hair, such as the devices described in Patent FR-2 586 913.

Using this device, it is possible to dye keratinous fibres using a method which comprises mixing the dyeing composition comprising at least one oxidation base of formula (I) with an oxidizing agent, and applying the mixture obtained to the keratinous fibres for a time sufficient to develop the desired colour.

The following non-limiting examples serve to illustrate the invention.

### EXAMPLES

### Example 1

### Synthesis of N-(4-hydroxy-3,3-dimethyl-2-oxo-2,3-dihydroindol-5-yl)acetamide

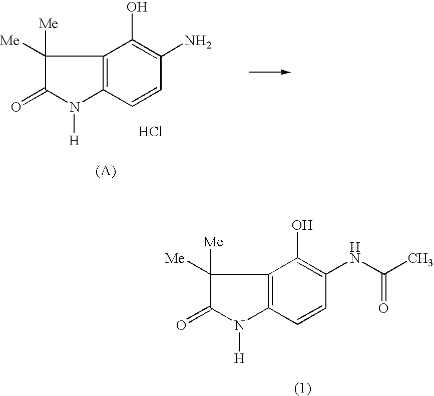

Compound (A) was prepared according to the synthesis described in U.S. Pat. No. 4,904,575. While keeping the temperature below 5° C., 8.1 ml of acetyl chloride was slowly added to a mixture comprising dimethylacetamide (50 ml), ethyl acetate (50 ml) and 20 g of compound (A). The reaction mixture was then mixed with ice-cold water and stirred. The product thus obtained was then filtered. After drying and washing in 100 ml of hot ethyl acetate, 23.1 g of product (1) was isolated by filtration and dried.

¹H NMR (300 MHz, CDCl₃): δ 1.30 (6H, s) 2.10 (2H, s) 6.33 (2H, d) 6.89 (2H, d) 9.73 (1H, s) 9.94 (1H, brs) 10.20 (1H, brs)

MS m/z 234 (M+)

Melting point>300° C.

### Example 2

### Synthesis of N-(8-chloro-4-hydroxy-3,3-dimethyl-2-oxo-2,3-dihydroincol-5-yl)acetamide

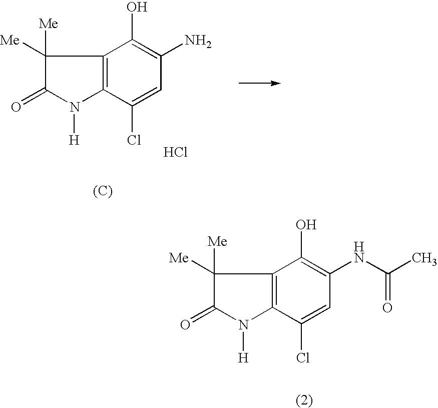

Compound (C) was prepared according to the synthesis described in U.S. Pat. No. 4,904,575. While keeping the temperature below 5° C., 6.9 ml of acetyl chloride was slowly added to a mixture comprising dimethylacetamide (50 ml), ethyl acetate (50 ml) and 20 g of compound (C). The reaction mixture was then mixed with ice-cold water and stirred. The product thus obtained was then filtered. After drying and washing in 100 ml of hot ethyl acetate, 22.7 g of product (2) was isolated by filtration and dried.

¹H NMR (300 MHz, CDCl₃): δ 1.32 (6H, s) 2.10 (2H, s) 7.06 (1H, s) 9.75 (1H, brs) 9.83 (1H, s) 10.62 (1H, s)

MS m/z 268 (M+)

Melting point=273-275° C.

### Example 3

### Synthesis of N-(8-chloro-5-hydroxy-4,4-dimethyl-2-oxo-1,2,3,4-tetrahydroquinolin-6-yl)acetamide

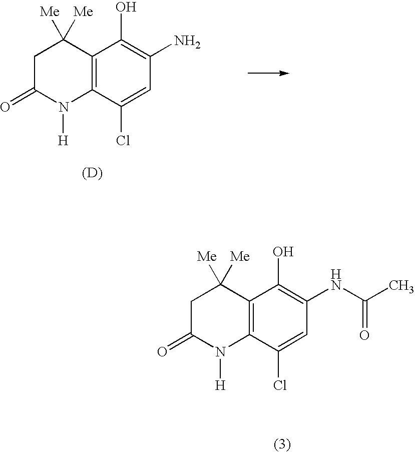

Compound (D) was prepared according to the syntheses described in U.S. Pat. No. 4,430,423. While keeping the temperature below 5° C., 3.9 ml of acetyl chloride was slowly added to 80 ml of methylacetamide comprising 12 g of compound (D). The reaction mixture was then mixed with ice-cold water and stirred. The product thus obtained was then filtered. After drying and washing in 60 ml of hot ethyl acetate, 13.0 g of product (3) was isolated by filtration and dried.

¹H NMR (300 MHz, CDCl₃): δ1.38 (6H, s) 2.09 (2H, s) 7.18 (1H, s) 9.20 (1H, brs) 9.39 (1H, s) 9.78 (1H, brs)

MS m/z 282 (M+)

Melting point=183-184° C.

### Example 4

### Synthesis of N-(5-hydroxy-4,4-dimethyl-2-oxo-1,2,3,4-tetrahydroquinolin-6-yl)trifluoromethylamide (4)

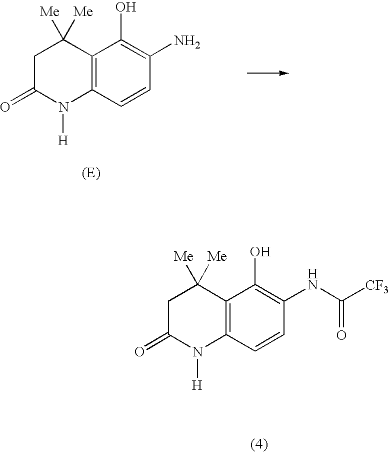

Compound (E) was prepared according to the synthesis described in U.S. Pat. No. 4,430,423. 7.5 ml of trifluoroacetic anhydride was slowly added to a solution comprising compound (E) in 40 ml of acetonitrile at room temperature. The reaction mixture was mixed with 100 ml of ethyl acetate, and then washed with water. The organic phase was dried over magnesium sulphate. The ethyl acetate was then evaporated under reduced pressure. After recrystallization from 50 ml of acetonitrile, 12.5 g of compound (4) was obtained and dried.

¹H NMR (300 MHz, CDCl₃): δ 1.35 (6H, s) 2.33 (2H, s) 6.39 (2H, d) 6.90 (2H, d) 9.10 (1H, brs) 10.40 (1H, brs)

MS m/z 300 (M+)

Melting point=126-129° C.

### Example 5

### Synthesis of N-(5-hydroxy-4,4-dimethyl-2-oxo-12,3,4-tetrahydroquinolin-6-yl)acetamide

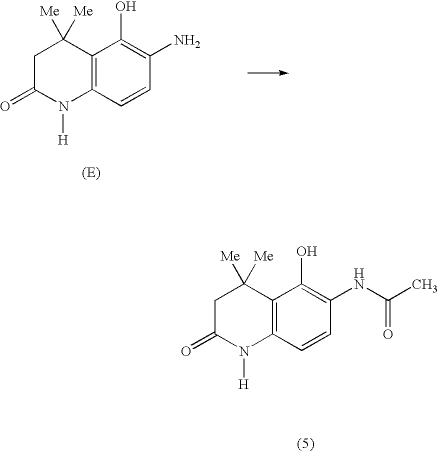

While keeping the temperature below 5° C., 8.1 ml of acetyl chloride was slowly added to a mixture comprising dimethylacetamide (50 ml), ethyl acetate (50 ml) and 19 g of compound (E). The reaction mixture was then mixed with ice-cold water and stirred. The product thus obtained was then filtered. After drying and washing in 100 ml of hot ethyl acetate, 22.5 g of product (5) was isolated by filtration and dried.

¹H NMR (300 MHz, CDCl₃): δ 1.32 (6H, s) 2.09 (3H, s) 2.31 (2H, s) 6.37 (2H, d) 6.88 (2H, d) 9.36 (1H, s) 9.89 (1H, brs) 10.00 (1H, brs)

MS m/z 247 (M+)

Melting point=211-212° C.

### Example 6

### Synthesis of N-(8-methoxy-5-hydroxy-4,4-dimethyl-2-oxo-1.2,3,4-tetrahydroquinolin-6-yl)acetamide

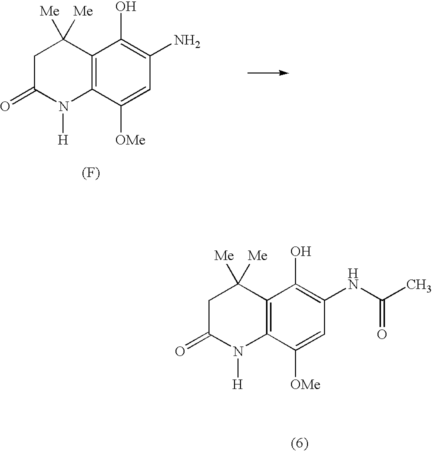

Compound (F) was prepared according to the synthesis described in U.S. Pat. No. 4,430,423. While keeping the temperature below 5° C., 4.2 ml of acetyl chloride was slowly added to a mixture comprising dimethylacetamide (20 ml), ethyl acetate (30 ml) and 10 g of compound (F). The reaction mixture was then mixed with ice-cold water and stirred. The product thus obtained was then filtered. After drying and washing in 100 ml of hot ethyl acetate, 10.7 g of product (6) were isolated by filtration and dried.

¹H NMR (300 MHz, CDCl₃): δ 1.37 (6H, s) 2.10 (3H, s) 2.31 (2H, s) 3.79 (3H, s) 6.72 (1H, s) 8.80 (1H, s) 8.88 (1H, brs) 9.90 (1H, brs)

MS m/z 277 (M+)

Melting point=176-178° C.

Examples of Dyes

The following non-limiting examples of the dyeing compositions disclosed herein were prepared:

| Examples | | | | |
|---|---|---|---|---|
| 1 | 2 | 3 | 4 | 5 |

| | | |
|---|---|---|
| N-(8-chloro-5-hydroxy-4,4- | 4 · 10⁻⁴ | mol |
| dimethyl-2-oxo-1,2,3,4- | | |
| tetrahydroquinolin-6- | | |
| yl)acetamide | | |

| | Examples | | | | | | | | | |
|---|---|---|---|---|---|---|---|---|---|---|
| | 6 | | 7 | | 8 | | 9 | | 10 | |
| N-(8-chloro-5-hydroxy-4,4- | 4 · 10⁻⁴ | mol | | | | | | | | |
| dimethyl-2-oxo-1,2,3,4- | | | | | | | | | | |
| tetrahydroquinolin-6- | | | | | | | | | | |
| yl)acetamide | | | | | | | | | | |
| N-(5-hydroxy-4,4-dimethyl- | | | 4 · 10⁻⁴ | mol | | | | | | |
| 2-oxo-1,2,3,4- | | | | | | | | | | |
| tetrahydroquinolin-6- | | | | | | | | | | |
| yl)acetamide | | | | | | | | | | |
| N-(8-methoxy-5-hydroxy- | | | | | 4 · 10⁻⁴ | mol | | | | |
| 4,4-dimethyl-2-oxo-1,2,3,4- | | | | | | | | | | |
| tetrahydroquinolin-6- | | | | | | | | | | |
| yl)acetamide | | | | | | | | | | |
| N-(7-chloro-4-hydroxy-3,3- | | | | | | | 4 · 10⁻⁴ | mol | | |
| dimethyl-2-oxo-2,3- | | | | | | | | | | |
| dihydroindol-5- | | | | | | | | | | |
| yl)acetamide | | | | | | | | | | |
| N-(4-hydroxy-3,3-dimethyl- | | | | | | | | | 4 · 10⁻⁴ | mol |
| 2-oxo-2,3-dihydroindol-5- | | | | | | | | | | |
| yl)acetamide | | | | | | | | | | |
| 7-amino-5-methyl-3- | | | | | | | | | | |
| aminopyrazolo[1,5- | | | | | | | | | | |
| a]pyrimidine.2HCl | | | | | | | | | | |
| 4-aminoaniline.2HCl | 4 · 10⁻⁴ | mol | 4 · 10⁻⁴ | mol | 4 · 10⁻⁴ | mol | 4 · 10⁻⁴ | mol | 4 · 10⁻⁴ | mol |
| Dye carrier (1) | (*) | | (*) | | (*) | | (*) | | (*) | |
| Demineralized water q.s. | 100 | g | 100 | g | 100 | g | 100 | g | 100 | g |
| | Examples | | | | | | | | | |
| | 11 | | 12 | | 13 | | 14 | | 15 | |
| N-(8-chloro-5-hydroxy-4,4- | 4 · 10⁻⁴ | mol | | | | | | | | |
| dimethyl-2-oxo-1,2,3,4- | | | | | | | | | | |
| tetrahydroquinolin-6- | | | | | | | | | | |
| yl)acetamide | | | | | | | | | | |
| N-(5-hydroxy-4,4-dimethyl- | | | 4 · 10⁻⁴ | mol | | | | | | |
| 2-oxo-1,2,3,4- | | | | | | | | | | |
| tetrahydroquinolin-6- | | | | | | | | | | |
| yl)acetamide | | | | | | | | | | |
| N-(8-methoxy-5-hydroxy- | | | | | 4 · 10⁻⁴ | mol | | | | |
| 4,4-dimethyl-2-oxo-1,2,3,4- | | | | | | | | | | |
| tetrahydroquinolin-6- | | | | | | | | | | |
| yl)acetamide | | | | | | | | | | |
| N-(7-chloro-4-hydroxy-3,3- | | | | | | | 4 · 10⁻⁴ | mol | | |
| dimethyl-2-oxo-2,3- | | | | | | | | | | |
| dihydroindol-5- | | | | | | | | | | |
| yl)acetamide | | | | | | | | | | |
| N-(4-hydroxy-3,3-dimethyl- | | | | | | | | | 4 · 10⁻⁴ | mol |
| 2-oxo-2,3-dihydroindol-5- | | | | | | | | | | |
| yl)acetamide | | | | | | | | | | |
| 4-(N,N-bis(2- | 4 · 10⁻⁴ | mol | 4 · 10⁻⁴ | mol | 4 · 10⁻⁴ | mol | 4 · 10⁻⁴ | mol | 4 · 10⁻⁴ | mol |
| hydroxyethyl)amino)- | | | | | | | | | | |
| aniline.2HCl | | | | | | | | | | |
| Dye carrier (1) | (*) | | (*) | | (*) | | (*) | | (*) | |
| Demineralized water q.s. | 100 | g | 100 | g | 100 | g | 100 | g | 100 | g |

| | |
|---|---|
| (*) Dye carrier (1) pH 9.5 | |
| DMSO | 0.18 g |
| Ethyl alcohol, 96° | 9.3 g |
| Methyl alcohol | 39.7 g |
| Acetic acid | 4.4 g |
| Sodium metabisulphite | 0.204 g |
| Pentasodium salt of | 1.1 g |
| diethylenetriaminopentaacetic acid as a 40% | |
| aqueous solution | |
| C₈-C₁₅ alkyl polyglucoside sold as a 60% solution | 5.3 g |
| under the name ORAMIXCG110 by the company | |
| SEPPIC | |
| Benzyl alcohol | 1.8 g |
| Polyethylene glycol containing 8 mol of ethylene | 2.7 g |
| oxide | |
| Buffer pH 9.5 NH₄Cl/ammonium hydroxide | 31.0 g |
| containing 20% NH₃ | |

At the time of use, the composition was mixed with a third of its weight of hydrogen peroxide at 20 volumes (6% by weight).

The mixture obtained was applied to a lock of grey hair which was 90% white. After an exposure time of 30 minutes, the lock was rinsed, washed with a standard shampoo, rinsed again and then dried.

Shades Observed:

| | Examples | | | | | | | |
|---|---|---|---|---|---|---|---|---|
| | 1 | 2 | 3 | 4 | 5 | 6 | 7 | 8 |
| Shade | violet- | violet- | violet- | violet | violet | blue | blue | blue |
| ob- | red | red | red | | | | | |
| served | | | | | | | | |

| | Examples | | | | | | |
|---|---|---|---|---|---|---|---|
| | 9 | 10 | 11 | 12 | 13 | 14 | 15 |
| Shade | violet- | violet- | blue | blue | blue | bluish | green |
| observed | red | red | | | | green | |

What is claimed is:

1. A dyeing composition comprising,
at least one cosmetic medium appropriate for dyeing keratinous fibres,
at least one oxidation base and
at least one coupler chosen from compounds of the following formula (I) and
any corresponding acid and base addition salts thereof 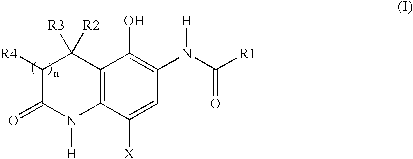
wherein
R₁ is a substituted or unsubstitued alkyl radical comprising from 1 to 10 carbon atoms, wherein the substituted alkyl radical comprises at least one radical chosen from halogen atoms, hydroxyl radicals, alkoxy radicals, carboxyl radicals, (C₁-C₄)alkylcarboxamido radicals, (NH₂—SO₂—) sulphonamido radicals, (C₁-C₄)alkylsulphonamido radicals, and NR₁₁R₁₂, wherein R₁₁ and R₁₂, which may be the same or different, are chosen from hydrogen atoms, and (C₁-C₂)alkyl radicals optionally substituted with at least one substituent chosen from hydroxyls and (C₁-C₂)alkoxys;
R₂, R₃ and R₄, which may be the same or different, are chosen from
(i) hydrogen atoms, and
(ii) (C₁-C₄)alkyl radicals optionally substituted with at least one radical chosen from hydroxyl, alkoxy, carboxyl, (C₁-C₄)alkylcarboxamido (AlkylNHCO—), (C₁-C₄)alkylsulphonyl (AlkylSO₂—), (C₁-C₄)alkylsulphoxide (AlkylSO—), sulphonamido (NH₂SO₂—), (C₁-C₄)alkylsulphonamido (AlkylNHSO₂—),sulphonic (—SO₃H), and NR₁₁R₁₂ radicals,
X is a hydrogen atom, a halogen atom, a substituted or unsubstituted (C₁-C₄)alkoxy radical, or a substituted or unsubstituted aryloxy radical, and
n is 0 or 1.

2. The composition according to claim 1, wherein R₁ is a C₁-C₆ alkyl radical.

3. The composition according to claim 1, wherein R₁ is an unsubstituted alkyl radical.

4. The composition according to claim 1, wherein R₂, R₃ and R₄, which may be identical or different, may be chosen from hydrogen atoms, C₁-C₄ alkyl radicals optionally substituted with at least one radical chosen from hydroxyl, methoxy, ethoxy, carboxyl, (C₁-C₄)alkylcarboxamido, sulphonamido and NR₁₁R₁₂ radicals.

5. The composition according to claim 4, wherein R₂, R₃ and R₄ are chosen from hydrogen atoms and unsubstituted alkyl radicals.

6. The composition according to claim 1, wherein X is a hydrogen atom, a chlorine atom or a substituted or unsubstituted alkoxy radical.

7. The composition according to claim 1, wherein the at least one coupler corresponds to the formula (I′) 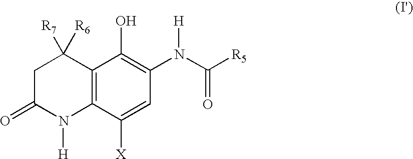
wherein R₅ is a C₁-C₆ alkyl radical, X is a hydrogen atom, a halogen atom or a (C₁-C₄)alkoxy radical, and R₆ and R₇, which may identical or different, are chosen from methyl and ethyl radicals.

8. The composition according to claim 7, wherein the coupler of formula (I′) is chosen from:
N-(8-chloro-5-hydroxy-4,4-dimethyl-2-oxo-1,2,3,4-tetrahydroquinolin-6-yl)acetamide,
N-(5-hydroxy-4,4-dimethyl-2-oxo-1,2,3,4-tetrahydroquinolin-6-yl)acetamide,
N-(8-chloro-5-hydroxy-4,4-dimethyl-2-oxo-1,2,3,4-tetrahydroquinolin-6-yl)propylamide,
N-(5-hydroxy-4,4-dimethyl-2-oxo-1,2,3,4-tetrahydroquinolin-6-yl)propylamide,
N-(8-methoxy-5-hydroxy-4,4-dimethyl-2-oxo-1,2,3,4-tetrahydroquinolin-6-yl)acetamide,
N-(8-methoxy-5-hydroxy-4,4-dimethyl-2-oxo-1,2,3,4-tetrahydroquinolin-6-yl)propylamide,
N-(8-chloro-5-hydroxy-2-oxo-1,2,3,4-tetrahydroquinolin-6-yl)acetamide,
N-(5-hydroxy-2-oxo-1,2,3,4-tetrahydroquinolin-6-yl)acetamide,
N-(8-chloro-5-hydroxy-2-oxo-1,2,3,4-tetrahydroquinolin-6-yl)propylamide,
N-(5-hydroxy-2-oxo-1,2,3,4-tetrahydroquinolin-6-yl)propylamide,
N-(8-methoxy-5-hydroxy-2-oxo-1,2,3,4-tetrahydroquinolin-6-yl)acetamide,
N-(8-methoxy-5-hydroxy-2-oxo-1,2,3,4-tetrahydroquinolin-6-yl)propylamide,
N-(7-chloro-4-hydroxy-3,3-dimethyl-2-oxo-2,3-dihydroindol-5-yl)acetamide,
N-(7-methoxy-4-hydroxy-3,3-dimethyl-2-oxo-2,3-dihydroindol-5-yl)acetamide,
N-(4-hydroxy-3,3-dimethyl-2-oxo-2,3-dihydroindol-5-yl)acetamide,
N-(7-chloro-4-hydroxy-3,3-dimethyl-2-oxo-2,3-dihydroindol-5-yl)propylamide,
N-(7-methoxy-4-hydroxy-3,3-dimethyl-2-oxo-2,3-dihydroindol-5-yl)propylamide,
N-(4-hydroxy-3,3-dimethyl-2-oxo-2,3-dihydroindol-5-yl)propylamide,
N-(7-chloro-4-hydroxy-2-oxo-2,3-dihydroindol-5-yl)acetamide,
N-(7-methoxy-4-hydroxy-2-oxo-2,3-dihydroindol-5-yl)acetamide,
N-(4-hydroxy-2-oxo-2,3-dihydroindol-5-yl)acetamide,
N-(7-chloro-4-hydroxy-2-oxo-2,3-dihydroindol-5-yl)propylamide,
N-(7-methoxy-4-hydroxy-2-oxo-2,3-dihydroindol-5-yl)propylamide, and
N-(4-hydroxy-2-oxo-2,3-dihydroindol-5-yl)propylamide.

9. The composition according to claim 1, wherein the coupler of formula (I) is chosen from N-(8-chloro-5-hydroxy-4,4-dimethyl-2-oxo-1,2,3,4-tetrahydroquinolin-6-yl)acetamide, N-(5-hydroxy-4,4-dimethyl-2-oxo-1,2,3,4-tetrahydroquinolin-6-yl)acetamide, N-(8-chloro-5-hydroxy-4,4-dimethyl-2-oxo-1,2,3,4-tetrahydroquinolin-6-yl)propylamide, N-(5-hydroxy-4,4-dimethyl-2-oxo-1,2,3,4-tetrahydroquinolin-6-yl)propylamide, N-(8-methoxy-5-hydroxy-4,4-dimethyl-2-oxo-1,2,3,4-tetrahydroquinolin-6-yl)acetamide, N-(8-methoxy-5-hydroxy-4,4-dimethyl-2-oxo-1,2,3,4-tetrahydroquinolin-6-yl)propylamide, N-(8-chloro-5-hydroxy-2-oxo-1,2,3,4-tetrahydroquinolin-6-yl)acetamide, N-(5-hydroxy-2-oxo-1,2,3,4-tetrahydroquinolin-6-yl)acetamide, N-(8-chloro-5-hydroxy-2-oxo-1,2,3,4-tetrahydroquinolin-6-yl)propylamide, N-(5-hydroxy-2-oxo-1,2,3,4-tetrahydroquinolin-6-yl)propylamide, N-(8-methoxy-5-hydroxy-2-oxo-1,2,3,4-tetrahydroquinolin-6-yl)acetamide, and N-(8-methoxy-5-hydroxy-2-oxo-1,2,3,4-tetrahydroquinolin-6-yl)propylamide.

10. The composition according to claim 1, wherein the amount of each of the couplers ranges from 0.001% to 10% by weight relative to the total weight of the dyeing composition.

11. The composition according to claim 1, wherein the at least one oxidation base is chosen from para-phenylenediamines, bisphenylalkylenediamines, para-aminophenols, bis-para-aminophenols, ortho-aminophenols, heterocyclic bases and addition salts thereof.

12. The composition according to claim 11, wherein the amount of each of the bases ranges from 0.001% to 10% by weight relative to the total weight of the dyeing composition.

13. A ready-to-use composition comprising at least one dyeing composition of claim 1 and at least one oxidizing agent.

14. The ready-to-use composition of claim 13, wherein the oxidizing agent is chosen from hydrogen peroxide, urea peroxide, alkali metal bromates, persalts, peracids and oxidase enzymes.

15. A method for the oxidation dyeing of keratinous fibres, comprising applying to the keratinous fibres a dyeing composition comprising
at least one cosmetic medium appropriate for dyeing keratinous fibres,
at least one oxidation base and
at least one coupler chosen from compounds of the following formula (I) and
any corresponding acid and base addition salts 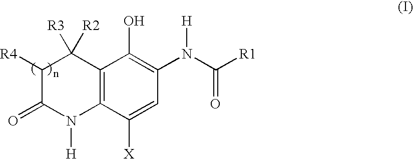
wherein
R₁ is a substituted or unsubstitued alkyl radical comprising from 1 to 10 carbon atoms, wherein the substituted alkyl radical comprises at least one radical chosen from halogen atoms, hydroxyl radicals, alkoxy radicals, carboxyl radicals, (C₁-C₄)alkylcarboxamido radicals, (NH₂—SO₂—) sulphonamido radicals, (C₁-C₄)alkylsulphonamido radicals, and NR₁₁R₁₂, wherein R₁₁ and R₁₂, which may be the same or different, are chosen from hydrogen atoms, and (C₁-C₂)alkyl radicals optionally substituted with at least one substituent chosen from hydroxyls and (C₁-C₂)alkoxys;
R₂, R₃ and R₄, which may be the same or different, are chosen from
(i) hydrogen atoms, and
(ii) (C₁-C₄)alkyl radicals optionally substituted with at least one radical chosen from hydroxyl, alkoxy, carboxyl, (C₁-C₄)alkylcarboxamido (AlkylNHCO—), (C₁-C₄)alkylsulphonyl (AlkylSO₂—), (C₁-C₄)alkylsulphoxide (AlkylSO—), sulphonamido (NH₂SO₂—), (C₁-C₄)alkylsulphonamido (AlkylNHSO₂—),sulphonic (—SO₃H), and NR₁₁R₁₂ radicals,
X is a hydrogen atom, a halogen atom, a substituted or unsubstituted (C₁-C₄)alkoxy radical, or a substituted or unsubstituted aryloxy radical, and
n is 0 or 1
in the presence of an oxidizing agent for a sufficient time to develop a desired colour.

16. A method according to claim 15, wherein the oxidizing agent is chosen from hydrogen peroxide, urea peroxide, alkali metal bromates, persalts, peracids and oxidase enzymes.

17. A multicompartment device comprising
a first compartment comprising a dyeing composition comprising,
at least one cosmetic medium appropriate for dyeing keratinous fibres,
at least one oxidation base and
at least one coupler chosen from compounds of the following formula (I) and
any corresponding acid and base addition salts 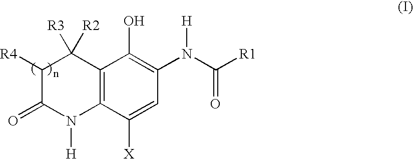
wherein
R₁ is a substituted or unsubstitued alkyl radical comprising from 1 to 10 carbon atoms, wherein the substituted alkyl radical comprises at least one radical chosen from halogen atoms, hydroxyl radicals, alkoxy radicals, carboxyl radicals, (C₁-C₄)alkylcarboxamido radicals, (NH₂—SO₂—) sulphonamido radicals, (C₁-C₄)alkylsulphonamido radicals, and NR₁₁R₁₂, wherein R₁, and R₁₂, which may be the same or different, are chosen from hydrogen atoms, and (C₁-C₂)alkyl radicals optionally substituted with at least one substituent chosen from hydroxyls and (C₁-C₂)alkoxys;
R₂, R₃ and R₄, which may be the same or different, are chosen from
(i) hydrogen atoms, and
(ii) (C₁-C₄)alkyl radicals optionally substituted with at least one radical chosen from hydroxyl, alkoxy, carboxyl, (C₁-C₄)alkylcarboxamido (AlkylNHCO—), (C₁-C₄)alkylsulphonyl (AlkylSO₂—), (C₁-C₄)alkylsulphoxide (AlkylSO—), sulphonamido (NH₂SO₂—), (C₁-C₄)alkylsulphonamido (AlkylNHSO₂—),sulphonic (—SO₃H), and NR₁₁R₁₂ radicals,
X is a hydrogen atom, a halogen atom, a substituted or unsubstituted (C₁-C₄)alkoxy radical, or a substituted or unsubstituted aryloxy radical, and
n is 0 or 1, and
a second compartment comprising an oxidizing agent.

18. The multicompartment device according to claim 17, wherein R₁ is a C₁-C₆ alkyl radical.

19. The multicompartment device according to claim 17, wherein R₁ is an unsubstituted alkyl radical.

20. The multicompartment device according to claim 17, wherein R₂, R₃ and R₄, which may be identical or different, may be chosen from hydrogen atoms, C₁-C₄ alkyl radicals optionally substituted with at least one radical chosen from hydroxyl, methoxy, ethoxy, carboxyl, (C₁-C₄)alkylcarboxamido, sulphonamido and NR₁₁R₁₂ radicals.

21. The multicompartment device according to claim 17, wherein R₂, R₃ and R₄ are chosen from hydrogen atoms and unsubstituted alkyl radicals.

22. The multicompartment device according to claim 17, wherein X is a hydrogen atom, a chlorine atom or a substituted or unsubstituted alkoxy radical.

23. The multicompartment device according to claim 17, wherein the at least one coupler corresponds to the formula (I′) 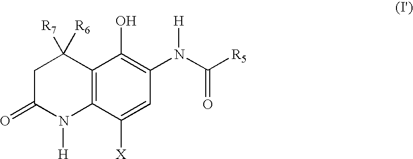
wherein R₅ is a C₁-C₆ alkyl radical, X is a hydrogen atom, a halogen atom or a (C₁-C₄)alkoxy radical, and R₆ and R₇, which may identical or different, are chosen from methyl and ethyl radicals.

24. The multicompartment device according to claim 17, wherein the coupler of formula (I′) is chosen from:
N-(8-chloro-5-hydroxy-4,4-dimethyl-2-oxo-1,2,3,4-tetrahydroquinolin-6-yl)acetamide,
N-(5-hydroxy-4,4-dimethyl-2-oxo-1,2,3,4-tetrahydroquinolin-6-yl)acetamide,
N-(8-chloro-5-hydroxy-4,4-dimethyl-2-oxo-1,2,3,4-tetrahydroquinolin-6-yl)propylamide,
N-(5-hydroxy-4,4-dimethyl-2-oxo-1,2,3,4-tetrahydroquinolin-6-yl)propylamide,
N-(8-methoxy-5-hydroxy-4,4-dimethyl-2-oxo-1,2,3,4-tetrahydroquinolin-6-yl)acetamide,
N-(8-methoxy-5-hydroxy-4,4-dimethyl-2-oxo-1,2,3,4-tetrahydroquinolin-6-yl)propylamide,
N-(8-chloro-5-hydroxy-2-oxo-1,2,3,4-tetrahydroquinolin-6-yl)acetamide,
N-(5-hydroxy-2-oxo-1,2,3,4-tetrahydroquinolin-6-yl)acetamide,
N-(8-chloro-5-hydroxy-2-oxo-1,2,3,4-tetrahydroquinolin-6-yl)propylamide,
N-(5-hydroxy-2-oxo-1,2,3,4-tetrahydroquinolin-6-yl)propylamide,
N-(8-methoxy-5-hydroxy-2-oxo-1,2,3,4-tetrahydroquinolin-6-yl)acetamide,
N-(8-methoxy-5-hydroxy-2-oxo-1,2,3,4-tetrahydroquinolin-6-yl)propylamide,
N-(7-chloro-4-hydroxy-3,3-dimethyl-2-oxo-2,3-dihydroindol-5-yl)acetamide,
N-(7-methoxy-4-hydroxy-3,3-dimethyl-2-oxo-2,3-dihydroindol-5-yl)acetamide,
N-(4-hydroxy-3,3-dimethyl-2-oxo-2,3-dihydroindol-5-yl)acetamide,
N-(7-chloro-4-hydroxy-3,3-dimethyl-2-oxo-2,3-dihydroindol-5-yl)propylamide,
N-(7-methoxy-4-hydroxy-3,3-dimethyl-2-oxo-2,3-dihydroindol-5-yl)propylamide,
N-(4-hydroxy-3,3-dimethyl-2-oxo-2,3-dihydroindol-5-yl)propylamide,
N-(7-chloro-4-hydroxy-2-oxo-2,3-dihydroindol-5-yl)acetamide,
N-(7-methoxy-4-hydroxy-2-oxo-2,3-dihydroindol-5-yl)acetamide,
N-(4-hydroxy-2-oxo-2,3-dihydroindol-5-yl)acetamide,
N-(7-chloro-4-hydroxy-2-oxo-2,3-dihydroindol-5-yl)propylamide,
N-(7-methoxy-4-hydroxy-2-oxo-2,3-dihydroindol-5-yl)propylamide, and
N-(4-hydroxy-2-oxo-2,3-dihydroindol-5-yl)propylamide.

25. The multicompartment device according to claim 17, wherein the coupler of formula (I) is chosen from N-(8-chloro-5-hydroxy-4,4-dimethyl-2-oxo-1,2,3,4-tetrahydroquinolin-6-yl)acetamide, N-(5-hydroxy-4,4-dimethyl-2-oxo-1,2,3,4-tetrahydroquinolin-6-yl)acetamide, N-(8-chloro-5-hydroxy-4,4-dimethyl-2-oxo-1,2,3,4-tetrahydroquinolin-6-yl)propylamide, N-(5-hydroxy-4,4-dimethyl-2-oxo-1,2,3,4-tetrahydroquinolin-6-yl)propylamide, N-(8-methoxy-5-hydroxy-4,4-dimethyl-2-oxo-1,2,3,4-tetrahydroquinolin-6-yl)acetamide, N-(8-methoxy-5-hydroxy-4,4-dimethyl-2-oxo-1,2,3,4-tetrahydroquinolin-6-yl)propylamide, N-(8-chloro-5-hydroxy-2-oxo-1,2,3,4-tetrahydroquinolin-6-yl)acetamide, N-(5-hydroxy-2-oxo-1,2,3,4-tetrahydroquinolin-6-yl)acetamide, N-(8-chloro-5-hydroxy-2-oxo-1,2,3,4-tetrahydroquinolin-6-yl)propylamide, N-(5-hydroxy-2-oxo-1,2,3,4-tetrahydroquinolin-6-yl)propylamide, N-(8-methoxy-5-hydroxy-2-oxo-1,2,3,4-tetrahydroquinolin-6-yl)acetamide, and N-(8-methoxy-5-hydroxy-2-oxo-1,2,3,4-tetrahydroquinolin-6-yl)propylamide.

26. The multicompartment device according to claim 17, wherein the oxidizing agent is chosen from hydrogen peroxide, urea peroxide, alkali metal bromates, persalts, peracids and oxidase enzymes.

27. A kit for dyeing keratinous fibres comprising
a dyeing composition comprising,
at least one cosmetic medium appropriate for dyeing keratinous fibres,
at least one oxidation base and
at least one coupler chosen from compounds of the following formula (I) and
any corresponding acid and base addition salts 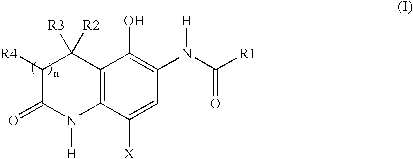
wherein
R₁ is a substituted or unsubstitued alkyl radical comprising from 1 to 10 carbon atoms, wherein the substituted alkyl radical comprises at least one radical chosen from halogen atoms, hydroxyl radicals, alkoxy radicals, carboxyl radicals, (C₁-C₄)alkylcarboxamido radicals, (N H₂—SO₂—) sulphonamido radicals, (C₁-C₄)alkylsulphonamido radicals, and NR₁R₁₂, wherein R₁₁ and R₁₂, which may be the same or different, are chosen from hydrogen atoms, and (C₁-C₂)alkyl radicals optionally substituted with at least one substituent chosen from hydroxyls and (C₁-C₂)alkoxys;
R₂, R₃ and R₄, which may be the same or different, are chosen from
(i) hydrogen atoms, and
(ii) (C₁-C₄)alkyl radicals optionally substituted with at least one radical chosen from hydroxyl, alkoxy, carboxyl, (C₁-C₄)alkylcarboxamido (AlkylNHCO—), (C₁-C₄)alkylsulphonyl (AlkylSO₂—), (C₁-C₄)alkylsulphoxide (AlkylSO—), sulphonamido (NH₂SO₂—), (C₁-C₄)alkylsulphonamido (AlkylNHSO₂—),sulphonic (—SO₃H), and NR₁₁R₁₂ radicals,
X is a hydrogen atom, a halogen atom, a substituted or unsubstituted (C₁-C₄)alkoxy radical, or a substituted or unsubstituted aryloxy radical, and
n is or 1, and
a second composition comprising an oxidizing agent.

28. The kit according to claim 27, wherein R₁ is a C₁-C₆ alkyl radical.

29. The kit according to claim 27, wherein R₁ is an unsubstituted alkyl radical.

30. The kit according to claim 27, wherein R₂, R₃ and R₄, wherein R₂, R₃ and R₄, which may be identical or different, may be chosen from hydrogen atoms, C₁-C₄ alkyl radicals optionally substituted with at least one radical chosen from hydroxyl, methoxy, ethoxy, carboxyl, (C₁-C₄)alkylcarboxamido, sulphonamido and NR₁₁R₁₂ radicals.

31. The kit according to claim 27, wherein R₂, R₃ and R₄ are chosen from hydrogen atoms and unsubstituted alkyl radicals.

32. The kit according to claim 27, wherein X is a hydrogen atom, a chlorine atom or a substituted or unsubstituted alkoxy radical.

33. The kit according to claim 27, wherein the at least one coupler corresponds to the formula (I′) 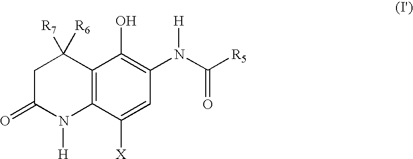
wherein R₅ is a C₁-C₆ alkyl radical, X is a hydrogen atom, a halogen atom or a (C₁-C₄)alkoxy radical, and R₆ and R₇, which may identical or different, are chosen from methyl and ethyl radicals.

34. The kit according to claim 27, wherein the coupler of formula (I′) is chosen from:
N-(8-chloro-5-hydroxy-4,4-dimethyl-2-oxo-1,2,3,4-tetrahydroquinolin-6-yl)acetamide,
N-(5-hydroxy-4,4-dimethyl-2-oxo-1,2,3,4-tetrahydroquinolin-6-yl)acetamide,
N-(8-chloro-5-hydroxy-4,4-dimethyl-2-oxo-1,2,3,4-tetrahydroquinolin-6-yl)propylamide,
N-(5-hydroxy-4,4-dimethyl-2-oxo-1,2,3,4-tetrahydroquinolin-6-yl)propylamide,
N-(8-methoxy-5-hydroxy-4,4-dimethyl-2-oxo-1,2,3,4-tetrahydroquinolin-6-yl)acetamide,
N-(8-methoxy-5-hydroxy-4,4-dimethyl-2-oxo-1,2,3,4-tetrahydroquinolin-6-yl)propylamide,
N-(8-chloro-5-hydroxy-2-oxo-1,2,3,4-tetrahydroquinolin-6-yl)acetamide,
N-(5-hydroxy-2-oxo-1,2,3,4-tetrahydroquinolin-6-yl)acetamide,
N-(8-chloro-5-hydroxy-2-oxo-1,2,3,4-tetrahydroquinolin-6-yl)propylamide,
N-(5-hydroxy-2-oxo-1,2,3,4-tetrahydroquinolin-6-yl)propylamide,
N-(8-methoxy-5-hydroxy-2-oxo-1,2,3,4-tetrahydroquinolin-6-yl)acetamide,
N-(8-methoxy-5-hydroxy-2-oxo-1,2,3,4-tetrahydroquinolin-6-yl)propylamide,
N-(7-chloro-4-hydroxy-3,3-dimethyl-2-oxo-2,3-dihydroindol-5-yl)acetamide,
N-(7-methoxy-4-hydroxy-3,3-dimethyl-2-oxo-2,3-dihydroindol-5-yl)acetamide,
N-(4-hydroxy-3,3-dimethyl-2-oxo-2,3-dihydroindol-5-yl)acetamide,
N-(7-chloro-4-hydroxy-3,3-dimethyl-2-oxo-2,3-dihydroindol-5-yl)propylamide,
N-(7-methoxy-4-hydroxy-3,3-dimethyl-2-oxo-2,3-dihydroindol-5-yl)propylamide,
N-(4-hydroxy-3,3-dimethyl-2-oxo-2,3-dihydroindol-5-yl)propylamide,
N-(7-chloro-4-hydroxy-2-oxo-2,3-dihydroindol-5-yl)acetamide,
N-(7-methoxy-4-hydroxy-2-oxo-2,3-dihydroindol-5-yl)acetamide,
N-(4-hydroxy-2-oxo-2,3-dihydroindol-5-yl)acetamide,
N-(7-chloro-4-hydroxy-2-oxo-2,3-dihydroindol-5-yl)propylamide,
N-(7-methoxy-4-hydroxy-2-oxo-2,3-dihydroindol-5-yl)propylamide, and
N-(4-hydroxy-2-oxo-2,3-dihydroindol-5-yl)propylamide.

35. The kit according to claim 27, wherein the coupler of formula (I) is chosen from N-(8-chloro-5-hydroxy-4,4-dimethyl-2-oxo-1,2,3,4-tetrahydroquinolin-6-yl)acetamide, N-(5-hydroxy-4,4-dimethyl-2-oxo-1,2,3,4-tetrahydroquinolin-6-yl)acetamide, N-(8-chloro-5-hydroxy-4,4-dimethyl-2-oxo-1,2,3,4-tetrahydroquinolin-6-yl)propylamide, N-(5-hydroxy-4,4-dimethyl-2-oxo-1,2,3,4-tetrahydroquinolin-6-yl)propylamide, N-(8-methoxy-5-hydroxy-4,4-dimethyl-2-oxo-1,2,3,4-tetrahydroquinolin-6-yl)acetamide, N-(8-methoxy-5-hydroxy-4,4-dimethyl-2-oxo-1,2,3,4-tetrahydroquinolin-6-yl)propylamide, N-(8-chloro-5-hydroxy-2-oxo-1,2,3,4-tetrahydroquinolin-6-yl)acetamide, N-(5-hydroxy-2-oxo-1,2,3,4-tetrahydroquinolin-6-yl)acetamide, N-(8-chloro-5-hydroxy-2-oxo-1,2,3,4-tetrahydroquinolin-6-yl)propylamide, N-(5-hydroxy-2-oxo-1,2,3,4-tetrahydroquinolin-6-yl)propylamide, N-(8-methoxy-5-hydroxy-2-oxo-1,2,3,4-tetrahydroquinolin-6-yl)acetamide, and N-(8-methoxy-5-hydroxy-2-oxo-1,2,3,4-tetrahydroquinolin-6-yl)propylamide.

36. The kit according to claim 27, wherein the oxidizing agent is chosen from hydrogen peroxide, urea peroxide, alkali metal bromates, persalts, peracids and oxidase enzymes.
